(19) United States
(12) Patent Application Publication
(10) Pub. No.: US 2020/0360520 A1
(43) Pub. Date: Nov. 19, 2020

(54) USE OF RIFAMYCIN-QUINOLIZIDONE COUPLING MOLECULE

(71) Applicant: TENNOR THERAPEUTICS (SUZHOU) LIMITED, Jiangsu (CN)

(72) Inventors: Zhenkun Ma, Jiangsu (CN); Ying Yuan, Jiangsu (CN); Yu Liu, Jiangsu (CN); Xiaomei Wang, Jiangsu (CN)

(73) Assignee: TENNOR THERAPEUTICS (SUZHOU) LIMITED, Jiangsu (CN)

(21) Appl. No.: 16/413,599

(22) Filed: May 15, 2019

(51) Int. Cl. A61K 47/54; A61P 31/04

(52) U.S. Cl. CPC A61K 47/545; A61K 31/395; A61P 31/04

(57) ABSTRACT

The present invention provides a use of a rifamycin-quinolizidone coupling molecule, or a stereoisomer, hydrate, deuterium-substituted form, ester, solvate, crystal form, metabolite, pharmaceutically acceptable salt or prodrug thereof in resisting nontuberculous mycobacteria. The rifamycin-quinolizidone coupling molecule has a structure shown in formula (I)

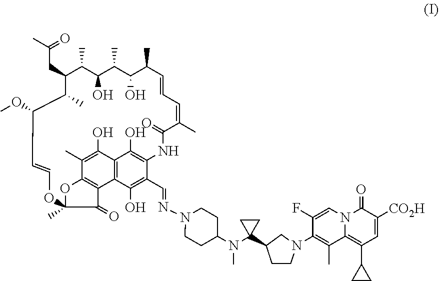 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](CC(C)=O)[C@@H]1C]

The rifamycin-quinolizidone coupling molecule, or the stereoisomer, hydrate, deuterium, ester, solvate, crystal form, metabolite, pharmaceutically acceptable salt or prodrug thereof may effectively against nontuberculous mycobacteria, and then may be used for treating infection caused by human nontuberculous mycobacteria.

### BACKGROUND

### 1. Field of the Invention

The present invention relates to a use of a rifamycin-quinolizidone coupling molecule, which belongs to the technical field of medicine.

### 2. Description of Related Art

Nontuberculous mycobacteria (NTM), also known as atypical mycobacteria, refer to mycobacteria except Mycobacterium tuberculosis complex (MTC) and Mycobacterium leprae. There are many methods for classifying mycobacteria. From the perspective of clinical guidance, useful information may be provided for medicine selection by simply classifying NTM into rapidly growing mycobacteria (RGM) and slowly growing mycobacteria (SGM). In view of the popularity of solid culture, this classification method may be implemented without special techniques and additional operations, thereby having strong practicality. Macroscopic colonies can be obtained by culturing RGM on a solid culture medium for 3-7 days, or by culturing SGM for several weeks. The most common clinically valuable RGM comprise Mycobacterium abscessus, Mycobacterium fortuitum and Mycodacterium chelonei, and thus RGM infection is usually treated with drugs such as macrolides, aminoglycosides and fluoroquinolones. The most common clinically valuable SGM comprise mycobacterium avium complex (MAC, mainly including Mycobacterium avium and Mycobacterium intracellulare), Mycobacterium kansasii and Mycobacterium xenopi, and thus SGM infection is usually treated with oral drugs such as macrolides and rifamycins, sometimes added with injectable antituberculosis drugs.

At present, the incidence of NTM infection has increased, which already becomes a major public health problem threatening human health in many countries. According to China's previous tuberculous epidemiological survey data, the NTM separation rate increased from 4.3% in 1979 to 11.1% in 2000, and 21% in 2010, showing a significant upward trend. NTM infection is mainly induced by Mycobacterium avium, Mycobacterium abscessus and Mycobacterium intracellulare in some provinces of China, but is mainly induced by Mycobacterium avium, Mycobacterium abscessus and Mycobacterium kansasii in the United States. Because mycobacteria inducing NTM infection are of different types, have different growth rates and characteristics, and have different drug resistance to antibacterial drugs, there is often a need to treat NTM infection with antibiotics for a long time, resulting low success rate and having significant side effects. Especially in recent years, the drug resistance of NTM to the existing drugs has increased, and the efficacy of the existing drugs has further decreased, so that the development of new therapeutic drugs and methods for treating NTM infection has become a major un-met need in global public health and clinical practice.

### SUMMARY

In view of the existing defects described in the prior art, an object of the present invention is to provide a use of a rifamycin-quinolizidone coupling molecule which can effectively inhibit and kill main pathogenic bacteria inducing NTM infection and then can be used for treating NTM infection.

The purpose of the present invention is realized by the following technical solution.

A use of a rifamycin-quinolizidone coupling molecule, or a stereoisomer, hydrate, deuterium-substituted form, ester, solvate, crystal form, metabolite, pharmaceutically acceptable salt or prodrug thereof in resisting NTM (i.e. nontuberculous mycobacteria), the rifamycin-quinolizidone coupling molecule having a structure shown in formula (I)

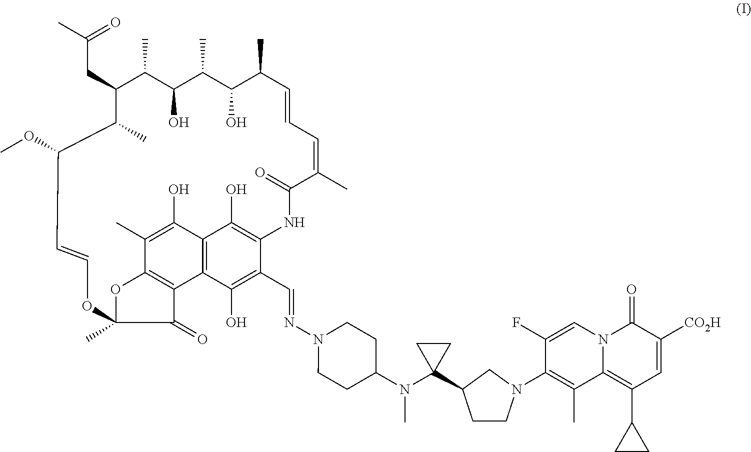 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](CC(C)=O)[C@@H]1C]

In the use above, preferably, the NTM may comprise one or more of Mycobacterium avium, Mycobacterium abscessus, Mycobacterium kansasii, Mycobacterium intracellulare, Mycobacterium massiliense, Mycodacterium chelonei and Mycobacterium fortuitum.

In the use, preferably, the NTM may comprise one or more of Mycobacterium avium, Mycobacterium abscessus and Mycobacterium kansasii.

The present invention further provides a use of the rifamycin-quinolizidone coupling molecule, or the stereoisomer, hydrate, deuterium-substituted form, ester, solvate, crystal form, metabolite, pharmaceutically acceptable salt or prodrug thereof in preparing a drug for treating a disease induced by human nontuberculous mycobacteria infection.

The present invention further provides a drug composition for resisting nontuberculous mycobacteria, wherein a component thereof may comprise the rifamycin-quinolizidone coupling molecule, or the stereoisomer, hydrate, deuterium-substituted form, ester, solvate, crystal form, metabolite, pharmaceutically acceptable salt or prodrug thereof.

The drug composition may comprise a combination of the rifamycin-quinolizidone coupling molecule and a conventional antibacterial drug in the art, a combination of the salt of the rifamycin-quinolizidone coupling molecule and a conventional antibacterial drug in the art, a combination of a mixture of the rifamycin-quinolizidone coupling molecule and the salt thereof and a conventional antibacterial drug in the art, and a combination of at least one of the stereoisomer, hydrate, deuterium-substituted form, ester, solvate, crystal form, metabolite, pharmaceutically acceptable salt or prodrug of the rifamycin-quinolizidone coupling molecule and a conventional antibacterial drug in the art.

The present invention further provides a use of the drug composition in preparing a drug for treating a disease induced by human nontuberculous mycobacteria infection.

The present invention has the following prominent effects.

The rifamycin-quinolizidone coupling molecule, or the stereoisomer, hydrate, deuterium-substituted form, ester, solvate, crystal form, metabolite, pharmaceutically acceptable salt or prodrug thereof of the present invention may effectively resist NTM, and then may be used for treating human NTM infection.

### DESCRIPTION OF THE EMBODIMENTS

To understand the technical features, purpose and advantageous effects of the present invention more clearly, the technical solution of the present invention is described in detail below, but cannot be understood as the limitation of the implementation scope of the present invention. The experimental methods described in the following embodiments are conventional methods unless otherwise specified; and the reagents and materials are commercially available unless otherwise specified.

### Embodiment 1

This embodiment provides a use of a rifamycin-quinolizidone coupling molecule in resisting NTM, the rifamycin-quinolizidone coupling molecule having a structure shown in formula (I)

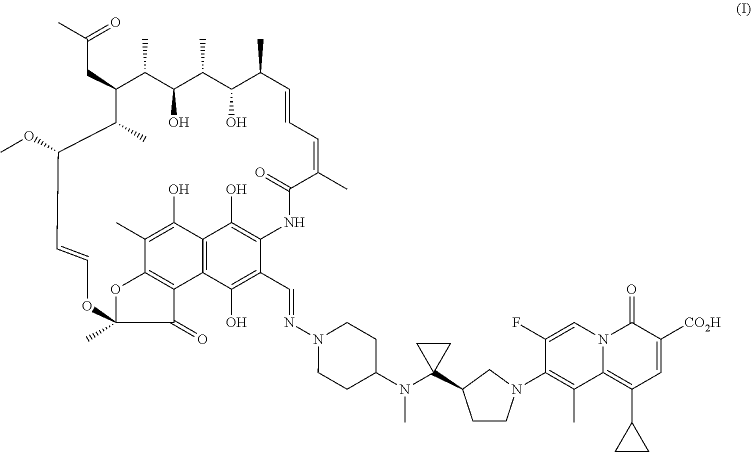 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](CC(C)=O)[C@@H]1C]

In this embodiment, an inhibitory test is performed on pathogenic bacteria, i.e. Mycobacterium avium, Mycobacterium abscessus and Mycobacterium kansasii belonging to the nontuberculous mycobacteria using the rifamycin-quinolizidone coupling molecule (formula I) of the present invention and positive controls, and then Minimum Inhibitory Concentration (MIC) and Minimum Bactericidal Concentration (MBC) are obtained. Clarithromycin, moxifloxacin, amikacin, rifampicin, rifabutin, ciprofloxacin and metronidazole are used as positive controls. The test strains are provided by KnowBio company, and the Chinese clinically isolated strains are derived from Shanghai Pulmonary Hospital.

The inhibitory test of this embodiment is performed by the micro broth dilution method using two different culture mediums: (1) MH micro broth dilution method: using MH culture medium (or culture solution), wherein the recommended concentration of calcium and magnesium ions in MH broth (cation-adjusted) is consistent with that in the Guideline of the Clinical and Laboratory Standards Institute (CLSI; M7-A7); and (2) 7H9 micro broth dilution method: using 7H9 culture medium (or culture solution, provided by Sigma-Aldrich). The reason of using two culture mediums, i.e. MH culture medium and 7H9 culture medium to perform composite screening is that the antimycobacterial compound shows different inhibitory activities in different liquid culture mediums, and embodiments of the present invention optimize the drug sensitivity test performed on NTM by using different broth culture mediums in the micro broth dilution method to make it closer to clinical conditions.

The method for testing MIC comprises the following steps.

Nontuberculous mycobacteria of rapidly growing mycobacteria (RGM) were grown on a 7H11 agar plate (provided by Sigma-Aldrich) in an air environment at 35-37° C. for about 3 days (depending on bacterial strains). Nontuberculous mycobacteria of slowly growing mycobacteria (SGM) were grown on a 7H11 agar plate (provided by Sigma-Aldrich) in an air environment at 37° C. for 21-30 days;

Certain colonies were selected from the agar plate, and then placed in the MH or 7H9 culture solution with 0.05% Tween-80. The selected colonies were cultured in an air environment at 35-37° C. for 3 days (rapidly growing) or 12 days (slowly growing) until the absorbance (OD value) thereof reached 0.08-0.1 (0.5 Mcfarland Standard). A bacterial suspension with the absorbance (OD600 value) of 0.08-0.1 (0.5 Mcfarland Standard) was prepared with normal saline.

180 μL of broth (MH or 7H9 culture solution) was added into the first column of holes of a 96-well plate. 100 μL of broth (MH or 7H9 culture solution) was added into other holes of the 96-well plate. The compound of formula I was formulated into 1.28 mg/mL of solution using DMSO to be tested in the range of 64-0.062 μg/mL immediately. 20 μL of compound was added into the first column of holes, and 100 μL thereof was taken for continuous dilution. Finally, 100 μL of nontuberculous mycobacteria strain suspension was added into all holes except the culture medium control holes, wherein different quality control reagents are added for different microorganisms. These holes include: 1) negative control holes only containing bacteria; 2) negative control holes only containing culture medium; 3) positive control holes containing clarithromycin and the like; and 4) control holes containing optional Escherichia coli.

For RGM, the OD value was assayed on the third day, and for SGM, the OD value was assayed on the twelfth day. Assay was performed by using the method of resazurin microtiter assay plate recommended by the Clinical and Laboratory Standards Institute. In short, the method was to add resazurin (7-hydroxy-3H-phenoxazin-3-one 10-oxide) into the 96-well plate. Resazurin is a blue dye, has weak fluorescence, may be irreversibly reduced to pink or highly red fluorescent dye; and may be used as a redox indicator when testing the MIC of living bacteria.

The method for testing MBC comprises the following steps.

A culture solution having a concentration equal to the MIC and a culture solution having a concentration higher than the MIC hole concentration (dilution of 0-1-2-3-4-5-6-7) were coated on 7H11 or MH agar plates in quadruplicate (four plates/holes), and then cultured in an air environment at 35-37° C. (depending on bacterial strains) to calculate CFU. wherein MIC₉₀ is the minimum drug concentration for inhibiting 90% of NTM isolated strains, and MBC₉₉ is the minimum drug concentration for killing 99.99% of initial bacteria.

The test results are shown in Tables 1-3:

TABLE 1
Minimum Inhibitory Concentration (MIC, mcg/mL) of Rifamycin-Quinolizidone
Coupling Molecule (Formula I) Against Nontuberculous Mycobacteria

| | 7H9 Broth | | | | MH Broth | | | |
|---|---|---|---|---|---|---|---|---|
| | Mycobacterium | | | | Mycobacterium | | | |
| | avium | | Mycobacterium | Mycobacterium | avium | | Mycobacterium | Mycobacterium |
| Compound | Smooth | Rough | abscessus | kansasii | Smooth | Rough | abscessus | kansasii |
| Compound I | 4 | >64 | 1 | 2 | 4 | >64 | 1 | 2 |
| Clarithromycin | 32 | >64 | 16 | 2 | >64 | >64 | 64 | 2 |
| Amikacin | 2 | >64 | 32 | 16 | 2 | 2 | 16 | 1 |
| Rifampicin | 0.5 | 8 | >64 | 16 | 1 | 8 | >64 | 16 |
| Ciprofloxacin | >64 | ND | 16 | >64 | >64 | ND | >64 | >64 |
| Metronidazole | >64 | ND | >64 | >64 | >64 | ND | >64 | >64 |

TABLE 2
Minimum Bactericidal Concentration (MBC, mcg/mL) of Rifamycin-Quinolizidone
Coupling Molecule (Formula I) Against Nontuberculous Mycobacteria

| | 7H9 Culture Medium | | | | MH Culture Medium | | | |
|---|---|---|---|---|---|---|---|---|
| | Mycobacterium | | | | Mycobacterium | | | |
| | avium | | Mycobacterium | Mycobacterium | avium | | Mycobacterium | Mycobacterium |
| Compound | Smooth | Rough | abscessus | kansasii | Smooth | Rough | abscessus | kansasii |
| Compound I | 16 | >64 | 1 | 2 | 16 | >64 | 1 | 2 |
| Clarithromycin | >64 | >64 | >64 | 64 | >64 | >64 | >64 | >64 |
| Amikacin | >64 | >64 | >64 | 64 | >64 | >64 | >64 | >64 |
| Rifampicin | 4 | >64 | >64 | 4 | 4 | ND | >64 | 4 |
| Ciprofloxacin | >64 | ND | >64 | >64 | >64 | ND | >64 | >64 |
| Metronidazole | >64 | ND | >64 | >64 | >64 | ND | >64 | >64 |

TABLE 3
Minimum Inhibitory Concentration (MIC, mcg/mL, MH Culture Medium) of Rifamycin-Quinolizidone
Coupling Molecule (Formula I) Against Isolated Strains from Chinese Hospital

| Clinically Isolated Strains | | Rifampicin | Moxifloxacin | Clarithromycin | Rifabutin | Metronidazole | Compound I |
|---|---|---|---|---|---|---|---|
| Mycobacterium | Avium (standard | 0.25 | 1 | 0.0625 | 0.0625 | 8 | 1 |
| avium | strain) | | | | | | |
| | Avium (S31) | 0.0625 | 8 | 0.125 | 0.0625 | 16 | 0.25 |
| | Avium (S123) | 8 | 0.5 | 0.5 | 0.0625 | 8 | 8 |
| | Avium (S559) | 0.125 | 1 | 0.25 | 0.0625 | 8 | 0.25 |
| | Avium (S568) | 4 | 0.5 | 4 | 0.0625 | 8 | 32 |
| | Avium (S577) | 0.125 | 0.5 | 0.25 | 0.0625 | 8 | 0.125 |
| | Avium (S597) | 8 | 2 | 1 | 0.0625 | 8 | >64 |
| | Avium (S602) | 2 | 4 | 1 | 0.0625 | 32 | 8 |
| Mycobacterium | Kansasii | 0.125 | 0.0625 | 0.0625 | 0.0625 | 8 | 0.0625 |
| kansasii | (standard strain) | | | | | | |
| | Kansasii (S9) | 0.125 | 0.0625 | 0.125 | 0.0625 | 8 | 0.25 |
| | Kansasii (S199) | 0.0625 | 0.0625 | 0.0625 | 0.0625 | 8 | 0.0625 |
| | Kansasii (S520) | 0.0625 | 0.0625 | 0.0625 | 0.0625 | 8 | 0.0625 |
| | Kansasii (S609) | 0.0625 | 0.0625 | 0.125 | 0.0625 | 8 | 0.0625 |
| Mycobacterium | Intracellulare | 0.25 | 0.25 | 0.0625 | 0.0625 | 8 | 0.5 |
| intracellulare | (standard strain) | | | | | | |
| | Intracellulare (S90) | 8 | 2 | 1 | 0.125 | 16 | 64 |
| | Intracellulare (S261) | 16 | 2 | 1 | 0.25 | 16 | 64 |
| | Intracellulare (S269) | 4 | 2 | 1 | 0.0625 | 16 | >64 |
| | Intracellulare (S290) | 4 | 2 | 1 | 0.0625 | 8 | 64 |
| | Intracellulare (S291) | 4 | 1 | 1 | 0.0625 | 8 | 64 |
| | Intracellulare (S298) | 4 | 2 | 2 | 0.5 | 16 | 64 |
| | intracellulare (S442) | 2 | 2 | 2 | 0.0625 | 8 | 32 |
| | Intracellulare (S541) | 16 | 64 | 64 | 0.125 | 16 | 32 |
| | Intracellulare (S623) | 8 | 2 | 4 | 0.25 | 8 | 64 |
| | Intracellulare (ZPS) | 2 | 1 | 1 | 0.0625 | 8 | 32 |
| Mycobacterium | Massiliense (D10) | >64 | 32 | 0.25 | >64 | 16 | >64 |
| massiliense | Massiliense (D14) | >64 | 32 | 0.0625 | 64 | 16 | >64 |
| | Massiliense (D18) | >64 | 8 | 0.0625 | 64 | 8 | >64 |
| Mycobacterium | Fortuitum | >64 | 1 | 32 | 4 | 16 | 64 |
| fortuitum | (standard strain) | | | | | | |
| | Fortuitum (S103) | >64 | 1 | >64 | 32 | 8 | >64 |
| | Fortuitum (S111) | 64 | 0.5 | 2 | 4 | 16 | >64 |
| Mycodacterium | Chelonei | >64 | 4 | 0.5 | 64 | 16 | 64 |
| chelonei | (standard strain) | | | | | | |

As shown in the above Tables 1, 2 and 3, the minimum inhibitory concentrations (MIC) of the rifamycin-quinolizidone coupling molecule (formula I) of the present invention against smooth type strain and rough type strain of Mycobacterium avium were similar to that of amikacin, and were superior to that of clarithromycin. The activity of the compound I against mycobacterium kansasii was much higher than that of rifampicin, ciprofloxacin and metronidazole, and was similar to that of other control compounds; and the activity of the compound against Mycobacterium intracellulare was weak. For the minimum bactericidal concentration (MBC), the bactericidal activity of the rifamycin-quinolizidone coupling molecule (formula I) against Mycobacterium kansasii was higher than or much higher than that of all other tested antibiotic drugs; and the rifamycin-quinolizidone coupling molecule also had an activity against smooth type strain of Mycobacterium avium. Meanwhile, the two different culture mediums had substantially identical test results, and can be suitable for clinical application. The results of research show that the rifamycin-quinolizidone coupling molecule of the present invention (formula I) had an effective and broad in-vitro activity against NTM, and thus may be used for treating human NTM infections.

In addition, the stereoisomer, hydrate, deuterium-substituted form, ester, solvate, crystal form, metabolite, pharmaceutically acceptable salt or prodrug of the rifamycin-quinolizidone coupling molecule of embodiments of the present invention may be used for resisting nontuberculous mycobacteria as well, and may be used for preparing a drug for treating a disease induced by human nontuberculous mycobacteria infections.

In another specific embodiment, the stereoisomer, hydrate, deuterium-substituted form, ester, solvate, crystal form, metabolite, pharmaceutically acceptable salt or prodrug of the rifamycin-quinolizidone coupling molecule may be combined with conventional antibacterial drugs in the art as well, to be used for treating a disease caused by NTM infections.

1. A method of inhibiting or killing nontuberculous mycobacteria, comprising:
contacting a rifamycin-quinolizidone coupling molecule having a formula (I) below or a pharmaceutically acceptable salt thereof with nontuberculous mycobacteria.

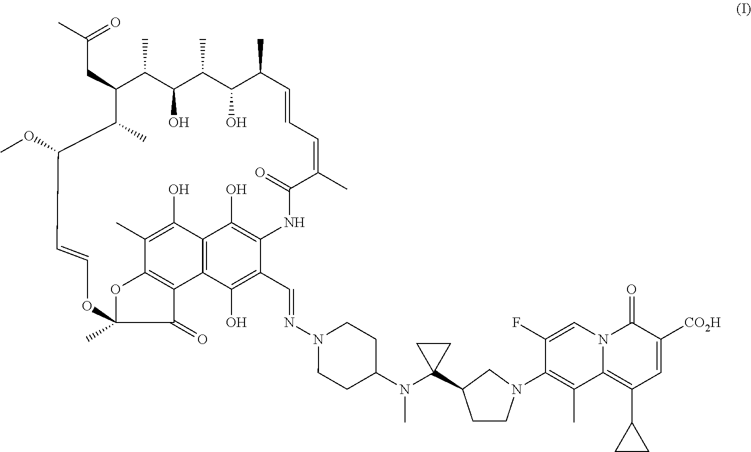 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](CC(C)=O)[C@@H]1C]

2. The method of claim 1, wherein the nontuberculous mycobacteria comprise Mycobacterium avium, Mycobacterium abscessus, Mycobacterium kansasii, Mycobacterium intracellulare, Mycobacterium massiliense, Mycodacterium chelonei, Mycobacterium fortuitum, or any combinations thereof.

3. The method of claim 1, wherein the nontuberculous mycobacteria comprise Mycobacterium avium, Mycobacterium abscessus, Mycobacterium kansasii, or any combinations thereof.

4. A method of treating infections of nontuberculous mycobacteria, comprising:
administrating a pharmaceutical composition comprising:
a pharmaceutical acceptable carrier; and
a pharmaceutical effective amount of a rifamycin-quinolizidone coupling molecule having a formula (I) below or a pharmaceutically acceptable salt thereof.

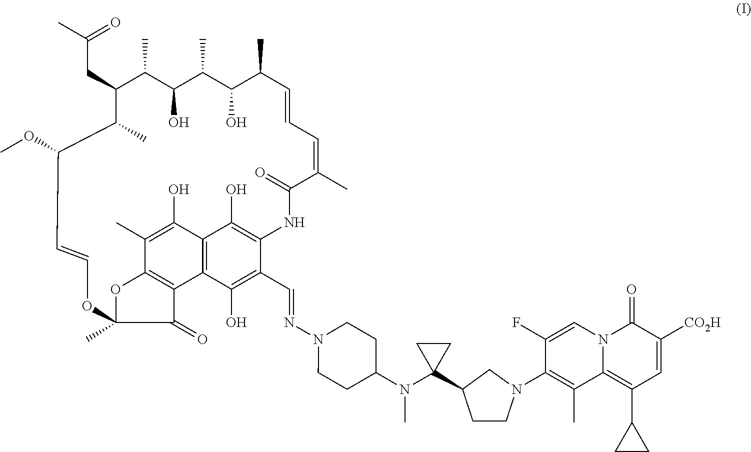 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](CC(C)=O)[C@@H]1C]

5. The method of claim 4, wherein the nontuberculous mycobacteria comprise Mycobacterium avium, Mycobacterium abscessus, Mycobacterium kansasii, Mycobacterium intracellulare, Mycobacterium massiliense, Mycodacterium chelonei, Mycobacterium fortuitum, or any combinations thereof.

6. The method of claim 4, wherein the nontuberculous mycobacteria comprise Mycobacterium avium, Mycobacterium abscessus, Mycobacterium kansasii, or any combinations thereof.

7. A pharmaceutical composition for treating nontuberculous mycobacteria infections, comprising:
a pharmaceutical acceptable carrier; and
a pharmaceutical effective amount of a rifamycin-quinolizidone coupling molecule having a formula (I) below or a pharmaceutically acceptable salt thereof.

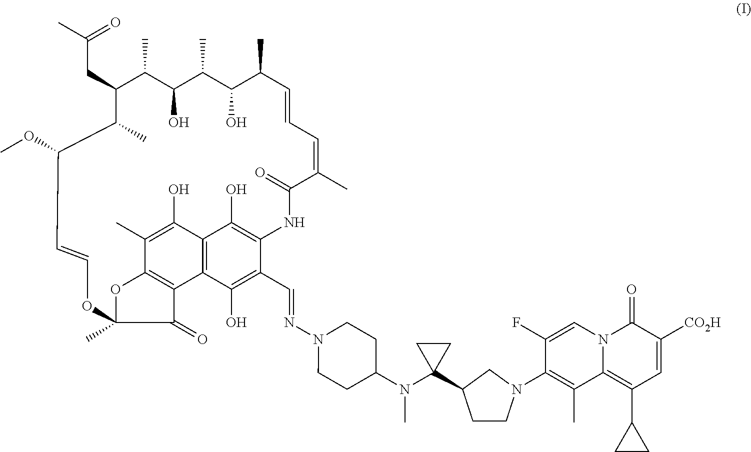 [SMILES: CO[C@H]1/C=C/O[C@@]2(C)Oc3c(C)c(O)c4c(O)c(c(/C=N/N5CCC(N(C)C6([C@@H]7CCN(c8c(F)cn9c(=O)c(C(=O)O)cc(C%10CC%10)c9c8C)C7)CC6)CC5)c(O)c4c3C2=O)NC(=O)/C(C)=C\C=C\[C@H](C)[C@H](O)[C@@H](C)[C@@H](O)[C@@H](C)[C@H](CC(C)=O)[C@@H]1C]

8. The pharmaceutical composition of claim 7, wherein the nontuberculous mycobacteria comprise Mycobacterium avium, Mycobacterium abscessus, Mycobacterium kansasii, Mycobacterium intracellulare, Mycobacterium massiliense, Mycodacterium chelonei, Mycobacterium fortuitum, or any combinations thereof.

9. The pharmaceutical composition of claim 7, wherein the nontuberculous mycobacteria comprise Mycobacterium avium, Mycobacterium abscessus, Mycobacterium kansasii, or any combinations thereof.
